(19) United States
(12) Patent Application Publication
(10) Pub. No.: US 2005/0020981 A1
(43) Pub. Date: Jan. 27, 2005

(54) Method and apparatus with a splittable hemostatic valve with a variable aperture

(76) Inventor: Paul A. Kurth, Santa Barbara, CA (US)

Correspondence Address: Daniel L. Dawes;MYERS DAWES ANDRDAS & SHERMAN LLP, Suite 1150, 19900 MacArthur Boulevard, Irvine, CA, 92612, US

(21) Appl. No.: 10/871,391

(22) Filed: Jun. 17, 2004

Related U.S. Application Data
(60) Provisional application No. 60/479,531, filed on Jun. 18, 2003.

(51) Int. Cl. A61M 5/178

(52) U.S. Cl. 604/167.06

(57) ABSTRACT

A separable hemostatic valve comprises a separable valve housing, a separable compressible valve in which an aperture is defined disposed in the valve housing, a means for varying the diameter of the aperture and/or compression of the valve around the aperture, and a means for separating the valve housing and valve into at least two parts. The means for varying the diameter of the aperture and/or compression of the valve comprises means for radially, laterally or longitudinally compressing the valve. The means for separating the valve housing and valve into at least two parts comprising means for slicing, splitting, peeling, tearing, parting or opening the valve housing and/or valve into parts.

### RELATED APPLICATIONS

The present application is related to U.S. Provisional Patent Application, Ser. No. 60/479,531, filed on Jun. 18, 2003, which is incorporated herein by reference and to which priority is claimed pursuant to 35 USC 119.

### BACKGROUND OF THE INVENTION

1. Field of the Invention

The invention relates to the field of endovascular devices and in particular to devices, such as introducers, which are combined with hemostatic valves.

2. Description of the Prior Art

Pacemaker leads currently being implanted into the left ventricle and coronary sinus typically have a diameter of 4 to 5 F and are provided with floppy tips. This combination between thinness and pliability often makes the insertion of these types of leads through a hemostatic valve provided on an introducer difficult, if not impractical. The problem becomes exacerbated when more than one lead must be disposed through the same hemostatic valve. The friction between the valve and the floppy lead is too great to allow advancement of the pliable portion of the lead through the valve. The aperture in the hemostatic valve cannot be permanently opened up or increased, because the valve must be able to provide a fluid-tight or air-tight seal not only around one or more pacemaker leads and floppy leads, but also around fine guidewires. Hence, the aperture in the valve must remain small and the elasticity of the valve must be high enough to insure a seal around both small diameter instruments as well as large diameter instruments.

To get around this difficulty, a practice has arisen in the profession to use “cheaters” or short introducers whose sole purpose is to provide a means by with such thin, floppy lead can be inserted through the hemostatic valve. These cheaters are nothing more than short tubes which are stiff enough to open up the hemostatic valve, which is an elastomeric plug with a slit or hole in it for the passage of the leads, wires and other instruments. The distal floppy portion of the lead is disposed through the cheater, which has a large enough inner diameter to allow the free passage of the floppy portion of the lead. More proximal portions of the floppy lead are stiffer, so that the cheater may be removed once these stiffer portions are advanced into the valve and the advance of the lead continued.

It is an object of the invention to provide a hemostatic valve in which the diameter of the aperture can be varied or the degree of elasticity or compression of the valve can be varied.

### BRIEF SUMMARY of the INVENTION

The invention is described in the illustrated embodiment as a separable hemostatic valve comprising a separable valve housing, a separable compressible valve member in which an aperture is defined and which valve member is disposed in the valve housing, a means for varying the diameter of the aperture and/or compression of the valve member around the aperture, and a means for separating the valve housing and valve member into at least two parts.

In one class of embodiments the means for varying the diameter of the aperture and/or compression of the valve member around the aperture comprises means for radially compressing the valve member. In another class of embodiments the means for varying the diameter of the aperture and/or compression of the valve member around the aperture comprises means for longitudinally compressing the valve member. In still another class of embodiments the means for varying the diameter of the aperture and/or compression of the valve member around the aperture comprises means for laterally compressing the valve member.

The means for separating the valve housing and valve member into at least two parts comprising means for slicing, splitting, peeling, tearing, parting or opening the valve housing and/or valve member into parts. However, it must be understood that the invention is not limited to these illustrated means, but must be understood to include all elements for performing the function of either varying the diameter of the aperture and/or compression of the valve member around the aperture.

In one embodiment the means for varying the diameter of the aperture and/or compression of the valve member around the aperture comprises a compression collar disposed about the valve housing, which is compressed radially by the compression collar. The compression collar is substantially fixed in radius and is coupled to the housing. The housing has an inner diameter and its walls have an increasing radius, so that longitudinal displacement of the collar with respect to the housing radially compresses the housing walls together and reduces the inner diameter of the housing thereby radially compressing the valve member therein and reducing the diameter of the aperture defined through the valve member.

In another embodiment the housing has a substantially constant inner diameter and the means for varying the diameter of the aperture and/or compression of the valve member around the aperture comprises a collar which is longitudinally disposable within the inner diameter of the housing. The collar has an inner diameter of decreasing magnitude as a function of distal position along the collar, so that longitudinal disposition of the collar into the housing compresses the valve member. In one version of this embodiment the collar has a stepped inner diameter.

In another embodiment the means for varying the diameter of the aperture and/or compression of the valve member around the aperture comprises a hydraulic collar disposed radially around the valve member. The hydraulic collar is inflatable to radially compress the valve member.

In still another embodiment the means for varying the diameter of the aperture and/or compression of the valve member around the aperture comprises a mechanical collar disposed radially around the valve member. The mechanical collar has a selectively controlled inner diameter which can be manipulated to radially compress the valve member.

In yet another embodiment the means for varying the diameter of the aperture and/or compression of the valve member around the aperture comprises at least one conical segment longitudinally disposable into the housing. The conical segment is disposed about the valve member and serves to radially compress the valve member when longitudinally disposed into the housing.

In another embodiment the means for varying the diameter of the aperture and/or compression of the valve member around the aperture comprises a plurality of nested conical segments longitudinally disposable into the housing. The nested conical segments are disposed about the valve member. In one version of the embodiment the valve member may also conical in shape. The conical segment may be oriented to have its least diameter proximally or distally positioned in the housing.

In one embodiment the valve member is a flexible funnel-shaped membrane defining the aperture therethrough. The housing is comprised of a proximal and distal section, which are rotatable relative to each other. The means for varying the diameter of the aperture and/or compression of the valve member around the aperture comprises a fixation of a proximal portion of the membrane to the proximal section of the housing and a fixation of a distal portion of the membrane to the distal section of the housing to allow the membrane to be twisted into a funnel shape of variable aperture by relative rotation of the proximal and distal sections of the housing with respect to each other. In such an embodiment the membrane may have surface modifications defined therein which tend to define how the membrane folds when twisted to assume the funnel shape of variable aperture. The proximal and distal sections of the housing are threaded together with a pitch matched to the lengthening and shortening of the membrane as it is twisted in either direction.

In yet another embodiment the valve member comprises a helical tube valve as defined below in this specification. The housing is comprised of a proximal and distal section. The means for varying the diameter of the aperture and/or compression of the valve member around the aperture comprises a means for telescopically displacing the proximal and distal sections with respect to each other.

The invention is also defined as a method of operating a hemostatic valve comprising the steps of inserting an elongate tool through an aperture defined in a valve member; applying a force to the valve member; varying the diameter of the aperture and/or force closing the valve member around the elongate tool disposed in the aperture; and separating the valve housing and valve member into at least two parts. The invention is also a method of using the various means disclosed above for varying the diameter of the aperture and/or force closing the valve member around the elongate tool disposed in the aperture.

While the apparatus and method has or will be described for the sake of grammatical fluidity with functional explanations, it is to be expressly understood that the claims, unless expressly formulated under 35 USC 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC 112 are to be accorded full statutory equivalents under 35 USC 112. The invention can be better visualized by turning now to the following drawings wherein like elements are referenced by like numerals.

### BRIEF DESCRIPTION OF THE DRAWINGS

FIG. 1 is a side cross sectional view of a first embodiment of the invention where a collar compresses a housing with a conical wall.

FIG. 2 is an end plan view of another embodiment of the invention where the valve is configured as an iris.

FIG. 3 is a side cross sectional view of another embodiment of the invention where a hydraulic collar is used to compress the valve.

FIG. 4 is a side cross sectional view of an embodiment of the invention where one or more nested conical segments are used to compress the valve.

FIG. 5 is a perspective view of a component of the embodiment of FIG. 4.

FIG. 6 is a side cross sectional view of an embodiment of the invention where the valve is a flexible membrane which is twisted.

FIG. 7 is a side cross sectional view of an embodiment of the invention where the valve is compressed by a conical inner surface of a telescoping housing.

FIG. 8 is a side cross sectional view of an embodiment of the invention where the valve is an elastomeric helical tube valve.

FIG. 9a is an end plan view of another embodiment of the invention where compression of the valve is realized by the lateral displacement of a pair of opposing wedges between the valve and the valve housing.

FIG. 9b is a side plan view of the embodiment of FIG. 9a. Also shown in FIGS. 9a and 9b are lines of weakening defined in the housing to allow separation of the valve housing into two parts.

FIG. 10 is a perspective view of the embodiment of FIG. 6 showing the lines of weakening defined in the housing to allow separation of the valve housing into two parts.

The invention and its various embodiments can now be better understood by turning to the following detailed description of the preferred embodiments which are presented as illustrated examples of the invention defined in the claims. It is expressly understood that the invention as defined by the claims may be broader than the illustrated embodiments described below.

### DETAILED DESCRIPTION OF THE PREFERRED EMBODIMENT

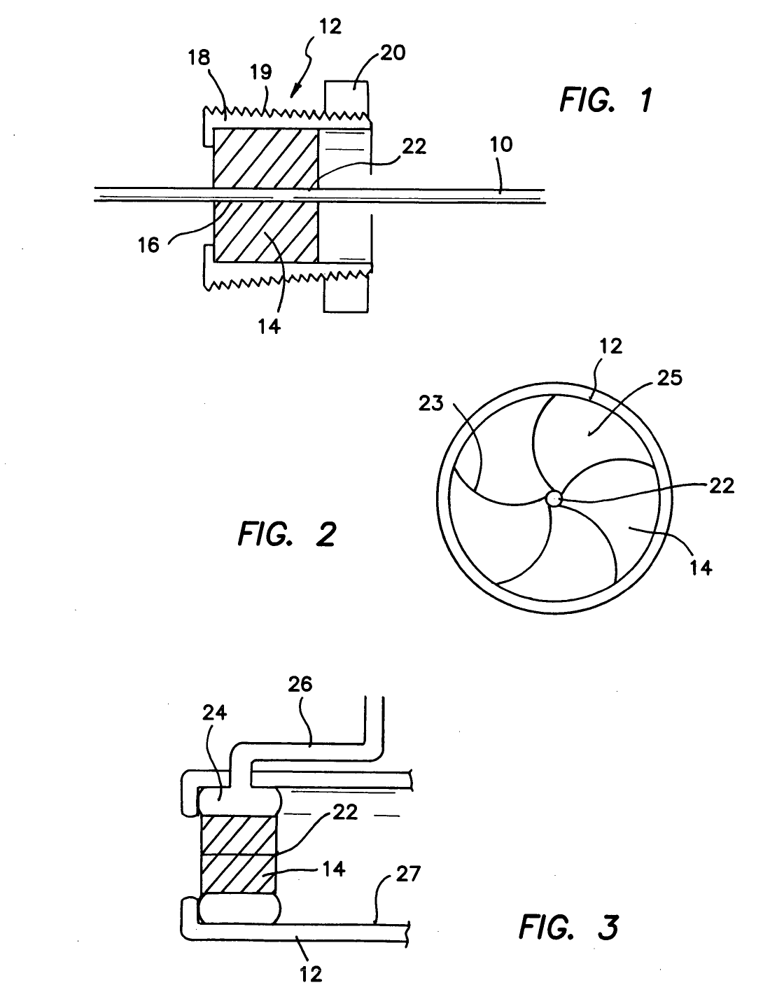

It must be borne in mind throughout the following disclosure that in each of the embodiments in FIGS. 1-5, valve housing 12 and/or its membrane or valve body 14 are openable, separable, tearable, sliceable, splittable or peelable by any one or more of a variety of means, such as described with respect to the separable valves disclosed below in the incorporated applications and patents.

As shown in FIG. 1 in side cross-sectional view a hemostatic valve housing 12 encapsulates or contains an elastomeric valve 14 through which an elgongate instrument 10 is disposed through an aperture or slit 22 defined through elastomeric valve 14. Instrument 10 may be any kind of vascular instrument, such as a guidewire, a pacemaker lead, a catheter, an introducer, a dilator or any other vascular tool or implement.

The proximal end of housing 12 is made so that it is compressible, such as being made of connecting sectors or portions of wedge shaped walls 18 having an exterior thread 19. In other words, walls 18 of housing 12 may be divided into two or more longitudinally separated portions, which are joined together at their distal end, such as a cylindrical section in which longitudinal slots have been cut or defined. These portions of walls 18 are capable then of being squeezed together to a predetermined degree.

A threaded compression collar 20 is threaded onto threads 19 of housing 12 so that when collar 20 is rotated to advance proximally, it compresses the wedge shaped walls 18 toward each other due to the increasing radial thickness of walls 18, thereby providing increased radial pressure on valve 14. Walls 18 are squeezed together as collar 20 rides up on the wedge shaped walls 18 as collar 18 is screw advanced in a proximal direction. Hence, valve 14 can be composed of material which is soft enough, and aperture 22 defined through valve 14 with enough clearance to allow a floppy lead tip to be disposed through aperture 22 and then when tighter sealing pressure is needed, as when a thinner guidewire is disposed therethrough, collar 20 can be tightened and the degree of compression of valve 14 about aperture 22 is increased.

It should also be understood that collar 20 may have a conical inner surface and walls 18 are cylindrical so that as collar 20 is threaded over walls 18 they are increasingly compressed. Housing 12 may be thin walled, made partially collapsible by means of longitudinal score or fold lines, or may have longitudinal slots defined in it to permit a limited degree of radial compression.

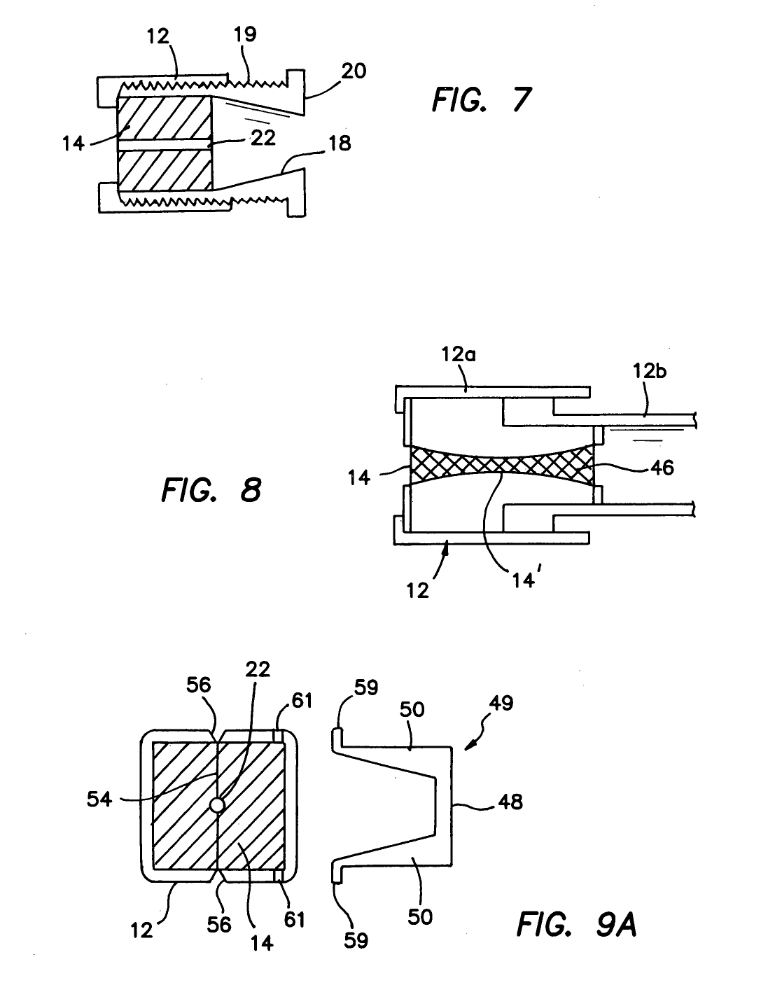

FIG. 7 depicts an alternative embodiment in which housing 12 is a right circular cylindrical segment and collar 20 is internally threaded into housing 12. Collar 20 in this embodiment has a conical inner surface 15 of decreasing diameter so that as it is longitudinally displaced over valve 14, valve 14 is radially compressed and aperture 22 reduced in diameter or at least closed with a greater degree of radially compressive force. Inner surface 15 may also have a compound stepped-down diameter to allow discrete amounts of compressive force to be applied to valve 14 over predetermined longitudinal distances of valve 14 according to the stepped nature of surface 15.

FIG. 2 is an end plan elevational view in which valve 14 is shown illustratively as being made of elastomeric iris segments and mounted in a housing 12, which includes a conventional iris mechanism to increase or decrease the diameter of the aperture 22 defined by the iris-defined aperture 22. The separations 23 defined through valve 14 which in combination form the iris about aperture 22 may be defined entirely or partly through valve 14 to allow a degree of longitudinal displacement of the various iris segments 25 lying between adjacent separations 23 to facilitate the decrease in diameter of aperture 22. Segments 25 may also be provided with an intervening lubrication, be composed of self-lubricating materials or have lubricating coatings which assist in the relative motion of the segments 25 with respect to each other with minimal friction. Segments 25 may also have stepped longitudinal profiles which allow each segment to radially overlap adjacent segments 25 when longitudinally displaced with respect to each other and to define a collectively smaller aperture 22.

FIG. 3 is a side cross-sectional view of another embodiment in which valve 14 and the pressure closing aperture 22 is controlled by means of a toroidal, hydraulically inflated collar 24 disposed between valve 14 and the inner walls 27 of housing 12. Fluid under pressure is communicated from an exterior pump or pressure source (not shown) through tubing 26. It must be understood that hydraulically inflated collar 24 can be replaced by a mechanical equivalent, namely a conventional iris ring in which the inner diameter of the ring decreases when rotated. The inner iris segments in such a ring then serve to radially compress valve 14 in a manner similar to the hydraulically inflated collar 24.

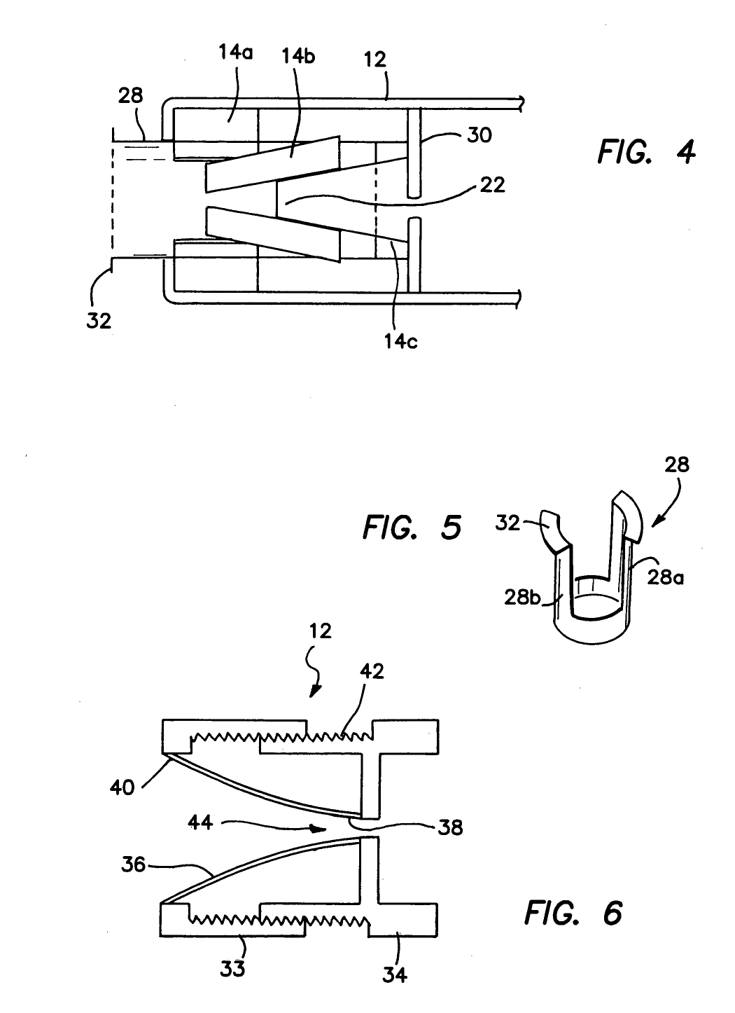

FIG. 4 is a side cross-sectional view of another embodiment in which valve 14 is comprised of a plurality of conical telescopic elastomeric segments 14a, 14b, 14c nested within each other. For the purposes of simplicity and clarity of illustration, FIG. 4 shows only three such segments 14a, 14b, 14c, but it is to be understood that any number of segments can be employed according to the teachings of the invention. In one configuration conical segments 14a, 14b, 14c are displaced each from the other so that aperture 22 defined in the last segment 14c is placed under minimal radial compression. However, split cylinder 28, described below in connection with FIG. 5, is connected to a slidable disk or piston 30 which abuts segment 14c and spans the inner diameter of housing 12. When cylinder 28 is pulled proximally using tabs 32, it de-telescopes or longitudinally collapses nested segments 14a, 14b and 14c together, which because of their conical shape, tend to increase the degree of compression on aperture 22 as the segments are brought into a more fully overlapping relationship.

The nested arrangement shown in FIG. 4 may easily be reversed. In other words, instead of having the segments 14a-14c oriented with their least diameter positioned toward the proximal end of housing 12, segments 14a-14c could also be oriented with their least diameter positioned toward the distal end of housing 12 with appropriate changes made in cylinder 28. In the reverse embodiment, piston 30 becomes a fixed wall in housing 12 and cylinder 28 becomes a movable piston operatively disposed in housing 12 to longitudinally distally displace segments 14a and/or 14b relative to segment 14c.

Furthermore, the valve, shown as segment 14c, is shown in the illustrated embodiment as conically shaped, but it need not be. Segment 14a may be cylindrical in profile as shown I FIG. 4 with only segment 14b being conical. Still further, segments 14a and 14b may be elastic in a manner which is the same or similar to segment 14c, which serves as the elastomeric valve and in which aperture 22 is defined, or segments 14a and 14b may be rigid. Finally, segments 14a, 14b and/or 14c may be made of self-lubricating material, be providing with intervening lubrication or coated with a lubricating material.

Split cylinder 28 as shown in FIG. 5 is rigid, thin walled and has opposing legs 28a and 28b which extend through arced cuts in segment 14a and which legs 28a and 28b are sealed by segment 14a. Proximal tabs 32 facilitate handling of cylinder 28.

Where there are only two segments 14a and 14b, there need be no longitudinal coupling, geared or measured engagement between the segments. However, where three of more segments 14a, 14b and 14c are used, each segment must be coupled or engaged to the adjacent one so then when cylinder 28 is distally moved, the plurality of segments can be safely telescoped to their expanded or extended configuration. Such couplings can be realized by tongue and groove connections between adjacent segments with end stops so that one segment is capable of pulling the next segment out with it.

While FIG. 2 is a multiple piece valve with iris portions similar in construction to a camera iris, FIG. 6 illustrates another embodiment of what could also be called an iris valve. A flexible, elastomeric, conical or funnel-shaped membrane 36 is provided inside a two-part valve housing 12, which is comprised of a proximal portion 32 and distal portion 34. The peripheral edge 40 of membrane 36 is connected or affixed to a proximal portion of proximal portion 33 of housing 12. The distal edge 38 of membrane 36 is connected or affixed to a distal portion of distal portion 34 of housing 12. Distal portion 34 and proximal portion 32 fit concentrically together and are adjustable coupled by means of mutually engaging threading 42. When membrane 36 is untwisted, it has a distal aperture 44 which is configured at its maximal diameter. When portions 32 and 34 of housing 12 are screwed together or rotated relative to each other, membrane 36 is twisted and as a result the aperture 44 will decrease in diameter. The effective thickness of membrane 36 may increase or decrease depending on the extent to which membrane 36 is lengthened or shortened by the rotation of portions 32 and 34. In addition, it must be understood that membrane 36 may be provided with folds or creases (not shown) which predetermine the manner in which membrane folds as it is twisted on itself.

The embodiment of FIG. 6 depicts the funnel shape in its untwisted configuration larger on the proximal end of housing 12 and smaller toward the distal end. However, it must be understood that the reverse orientation is also contemplated, namely that the funnel shape in its untwisted configuration smaller on the proximal end of housing 12 and larger toward the distal end.

FIG. 8 is a side cross-sectional view of another embodiment in which housing 12 is comprised of a proximal portion 12a and a distal portion 12bwhich are telescopically engaged with each other. Sections 12a and 12b may be engaged with each other by a key and groove combination, threading, rack and pinion, or simply by friction. Valve 14 in this case is a braid reinforced elastomeric tube. The braid 46 embedded in or around the elastomeric tube 14′ is comprised to two sets of helical windings comprised of substantially nonextensible fibers. The sets of helical windings are wound in opposite directions and may be loosely overlaid or wound in a spaced manner so that when the tension on tube 14′ is increased by relative telescope movement or force applied to sections 12a and 12b, the tightness of the helical windings changes, much like a child's Chinese finger toy lock, to reduce the diameter of tube 14′. The elastomeric nature of tube 14′ accommodates the compression or expansion which windings 46 apply to it to provide a greater or lesser degree of radially compressive force on any elongate object disposed through tube 14′. Thus, in this embodiment the actual inner diameter of tube 14′ may not change to greatly as does the degree of radially compressive force applied to an inserted object. By this means and selection of the softness of the elastomeric material comprising tube 14′, thinner or thicker elongate instrument(s) may be accommodated through tube 14′ and still sealed in a fluid tight manner. For the purposes of this specification, this type of a valve will be defined as “helical tube valve”.

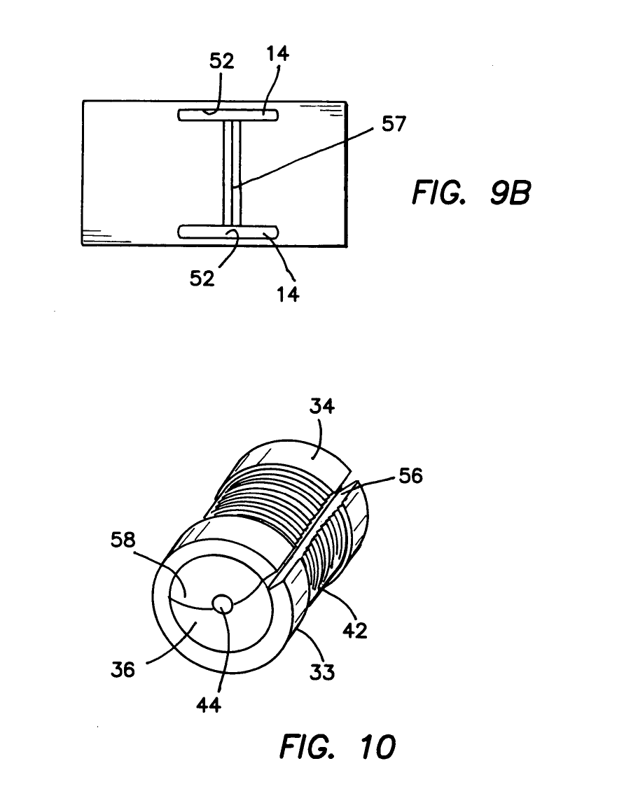

FIGS. 9a and 9b are a front plan exploded view and a side plan view of the another embodiment in which valve 14 and its housing 12 are rectangular or prismatic in shape. Slots 52 are defined through housing 12 to allow the insertion of wedged shaped legs 50 of a compression clip 49 into housing 12 above and below the upper and lower surface of valve 14 between valve 14 and the adjacent wall of housing 12. Clip 49 is a U-shaped rigid clip with two opposing wedged-shaped legs 50 connected by a spine 48. It is of course possible to modify the design of FIGS. 9a and 9b and use a clip 49 having a single leg 50, if desired. In either case the lateral insertion of legs 50 between valve 14 and housing 12 through slots 52 serve to compress valve 14 and close or narrow central aperture 22 defined therethrough. The degree of insertion of clip 49 into housing 12 determines the degree of compression of aperture 22. Many other means can be equivalently employed to provide lateral compression of valve 14 other the clip and housing combination as described in this embodiment.

In the embodiment of FIGS. 9a and 9b it is assumed that the instrument used with the hemostatic valve is longitudinally inserted through the valve. However, the embodiment of FIGS. 9a and 9b could also be modified to allow the elongate instrument to be laterally inserted through housing 12 by defining a longitudinal slot through the side wall of housing 12 and splitting valve 14 along plane 54 to allow lateral passage of the instrument through housing 12 and valve 14. Clip 49 may then be used with such a lateral-access hemostatic valve in the same way as an embodiment permitting only longitudinal access.

Finally, it must again be borne in mind that valve housing 12 and membrane 36 are separable, sliceable, splittable or peelable by any one of a variety of means such as described in the separable valves disclosed below in the incorporated patents and applications. Many other devices can be imagined for varying the compression on valve 14 or the diameter of aperture 22 consistent with the spirit and scope of the invention and the invention is not limited by the examples discussed here.

While the illustrated embodiment has been described as a valve with various means for varying the aperture of the hemostatic valve, it must be expressly understood that the disclosed valve is used in combination with any sliceable, splittable, peelable, tearable or separable catheter or introducer now known or later devised, as well as catheters or introducers, which cannot be separated in any of these manners. In addition, whether or not a catheter or introducer is associated with the hemostatic valve of the invention, and if so, whether or not the catheter or introducer is separable or not, together with or separately from the hemostatic valve, is all expressly included within the scope of the disclosed invention.

For example, splittable slidable, openable, side-access valves are included of the type as disclosed in Lee, “Splittable Hemostatic Valve and Sheath and the Method for Using the Same”, U.S. Pat. No. 5,125,904 (1992) and U.S. Pat. No. 5,312,355 (1994); Pohndorf, “Sheath Introducer With Valve That Splits,” U.S. Pat. No. 5,613,953 (1997), and “Splittable Lead Introducer With Mechanical Opening Valve,” U.S. Pat. No. 5,441,504 (1995), which are incorporated herein by reference.

Catheters and introducers are included of the type as disclosed in: Kurth, “Permanent Catheter with an Exterior Balloon Valve and Method of Using the Same,” U.S. Pat. No. 5,792,118 (1998), “Method and Apparatus for Insertion of Elongate Instruments Within a Body Cavity,” U.S. patent application Ser. No. 09/708,150 (2000), “A Temporarily Secured Guidewire and Catheter for Use in the Coronary Venous System and Method of Using the Same,” U.S. patent application Ser. No. 10/365,890 (2003), “A Method and Apparatus for a Suction-Anchored Introducer for Pacemaker Implantation, ” U.S. Provisional Patent Application Ser. No. 60/464,437 (2003), “Method and Apparatus for Implantation of Left Ventricular pacing Leads Between the Epicardium and Pericardium, ” U.S. Provisional Patent Application Ser. No. 60/426,773 (2002), and “A Tool for Placement of Dual Angioplasty Wires in the Coronary Sinus Vasculature,” U.S. Provisional Patent Application Ser. No. 60/408,385 (2002); Worley et. al., “Introducer for Accessing the Coronary Sinus of a Heart,” U.S. patent application Ser. No. 10/139,551 (2002), “A Telescopic, Peel-Away Introducer and Method of Using the Same,” U.S. patent application Ser. No. 10/139,554 (2002), “A Telescopic, Peel-Away Introducer and Method of Using the Same,” U.S. patent application Ser. No. 10/139,554 (2002), “A Telescopic Introducer with a Compound Curvature for Inducing Alignment and Method of Using the Same”, U.S. patent application Ser. No. 10/202,158; and Kurth et.al., “Introducer and Hemostatic Valve Combination and Method of Using the Same, ” U.S. patent application Ser. No. 10/234,686 (2002), “A Compression Fitting for an Introducer Coupled to a Hemostatic Valve,” U.S. patent application Ser. No. 10/277,476 (2002), which are all incorporated herein by reference.

Consider how the approaches of FIGS. 1-9b and other embodiments made separable. For the purposes of this specification and its claims, the term “separable” or “separating” is defined to mean any method, mechanism, scheme or device by in which the valve and/or any sheath or introducer to which it may be attached can be divided into two or more parts, including but not limited to any sliceable, splittable, peelable, tearable, separable, partable or openable mechanism now known or later devised. The variable aperture hemostatic valves of the embodiments of FIGS. 1, 2, 6, or 7 each contemplate some type of rotation of various elements to varying the aperture size or force of compression on the aperture 22. The variable aperture hemostatic valves of the embodiments of FIGS. 4, 5, and 8 each contemplate some type of longitudinal displacement of various elements to varying the aperture size or force of compression on the aperture 22. The variable aperture hemostatic valves of the embodiments of FIGS. 3, 9a, and 9b each contemplate the activation of some type of compression element for varying the aperture size or force on the aperture 22, namely a hydraulic force in the case of FIG. 3 and a mechanical wedge in the case of FIGS. 9a and 9b.

In the last two classes of devices, namely FIGS. 3, 4, 5, 8, 9a, and 9b where the components are operated by means of relative linear displacement, the hemostatic valve can be made separable by conventional means, such as weakened lines of separation 56 molded into the walls of housing 12 as shown illustratively in FIG. 9a and 9b. Lines of separation 56 are defined in the wall of housing 12 which are adjacent or parallel to legs 50 of clip 49, so that housing 12 can be separated without disengaging clip 49 off valve 14. Valve 14 is similarly partially or entirely cut or made in halves as shown in FIG. 9a by cut line 54. Housing 12 is manually grasped by means of tabs or other conventional means, and can be separated along lines 56 carrying the halves of valve 14 with each half of housing 12 or allowing valve 14 to be independently separated. Similarly, an introducer or sheath attached to housing 12 is separated along with it or separately from it on similar lines of separation, which are well known to the art.

Alternatively, and preferably in addition to separation lines 56, diametrically opposed separation lines 57 may be defined in housing 12 which are defined vertically between slots 52. This allows the front and back half of housing 12 to be separated from clip 49 even after being longitudinally separated.

Furthermore, clip 49 is captured by detent pins 59 on the ends of legs 50 which snap into holes 61 defined in the adjacent walls of housing 12 near slots 52 in the adjacent wall of housing 12. In this manner clip 49 is not left free in the operating theater and is retained at all times to housing 12. Clip 49 can be released from housing 12 and laterally displaced inwardly across valve 14 to be operated by simultaneously depressing detent pins 59 and laterally urging clip 49 into housing 12.

In the class of devices of FIGS. 1, 2, 6, or 7 where the components are operated by means of relative rotation, the hemostatic valve can be made separable by defining a weakened line or lines of separation of the relatively rotating parts, which lines are then aligned and both components separated in the same manner as in the embodiments above. The degree compressibility of valve 14 is chosen by appropriate empirical selection of materials, such that rotation of the parts and further compression of valve 14 beyond initial closure is always possible. For example, the embodiment of FIG. 6 is shown in perspective view in FIG. 10 where weakened lines of separation 56 has been defined in both proximal portion 33 and distal portion 34 of housing 12. Only one line 56 is shown in FIG. 10, but a diametrically opposing line 56 is also defined in proximal portion 33 and distal portion 34 allowing housing 12 to be separated. The line 56 in proximal portion 33 and distal portion 34 are shown in FIG. 10 as being aligned, but in general the angular alignment of portions 33 and 34 is arbitrary. Membrane 36 similarly may include weakened lines of separation 58 to facilitate its separation. In the event that the deformability of valve 14 in any embodiment would not permit up to almost 180° or relative rotation, separation lines 56 could be defined into portions 33 and 34 every 90° or less until the deformability of valve 14 was accommodated. Still further, there is nothing to prohibit a two step separation process, which does not require alignment of the separation lines 56 on portions 33 and 34, but contemplates a first step of separating portion 33 and then the step of separating portion 34.

Many alterations and modifications may be made by those having ordinary skill in the art without departing from the spirit and scope of the invention. For example,

Therefore, it must be understood that the illustrated embodiment has been set forth only for the purposes of example and that it should not be taken as limiting the invention as defined by the following claims. For example, notwithstanding the fact that the elements of a claim are set forth below in a certain combination, it must be expressly understood that the invention includes other combinations of fewer, more or different elements, which are disclosed in above even when not initially claimed in such combinations.

The words used in this specification to describe the invention and its various embodiments are to be understood not only in the sense of their commonly defined meanings, but to include by special definition in this specification structure, material or acts beyond the scope of the commonly defined meanings. Thus if an element can be understood in the context of this specification as including more than one meaning, then its use in a claim must be understood as being generic to all possible meanings supported by the specification and by the word itself.

The definitions of the words or elements of the following claims are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptionally equivalent, what can be obviously substituted and also what essentially incorporates the essential idea of the invention.

1. A separable hemostatic valve comprising:
a separable valve housing;
a separable compressible valve member in which an aperture is defined and which valve member disposed in the valve housing;
means for varying the diameter of the aperture and/or compression of the valve member around the aperture; and
means for separating the valve housing and valve member into at least two parts.

2. The hemostatic valve of claim 1 where the means for varying the diameter of the aperture and/or compression of the valve member around the aperture comprises means for radially compressing the valve member.

3. The hemostatic valve of claim 1 where the means for varying the diameter of the aperture and/or compression of the valve member around the aperture comprises means for longitudinally compressing the valve member.

4. The hemostatic valve of claim 1 where the means for varying the diameter of the aperture and/or compression of the valve member around the aperture comprises means for laterally compressing the valve member.

5. The hemostatic valve of claim 1 where the means for separating the valve housing and valve member into at least two parts comprising means for slicing, splitting, peeling, tearing, parting or opening the valve housing and/or valve member into parts.

6. The hemostatic valve of claim 1 where the means for varying the diameter of the aperture and/or compression of the valve member around the aperture comprises a compression collar disposed about the valve housing, which is compressed radially by the compression collar.

7. The hemostatic valve of claim 6 where the compression collar is substantially fixed in radius and is coupled to the housing, and where the housing has an inner diameter and walls which are of increasing radius, so that longitudinal displacement of the collar with respect to the housing radially compresses the housing walls together and reduces the inner diameter of the housing thereby radially compressing the valve member therein and reducing the diameter of the aperture defined through the valve member.

8. The hemostatic valve of claim 1 where the housing has a substantially constant inner diameter and where the means for varying the diameter of the aperture and/or compression of the valve member around the aperture comprises collar longitudinally disposable within the inner diameter of the housing and where the collar has an inner diameter of decreasing magnitude as a function of distal position along the collar, so that longitudinal disposition of the collar into the housing compresses the valve member.

9. The hemostatic valve of claim 8 where the collar has a stepped inner diameter.

10. The hemostatic valve of claim 1 where the means for varying the diameter of the aperture and/or compression of the valve member around the aperture comprises an hydraulic collar disposed radially around the valve member, the hydraulic collar being inflatable to radially compress the valve member.

11. The hemostatic valve of claim 1 where the means for varying the diameter of the aperture and/or compression of the valve member around the aperture comprises a mechanical collar disposed radially around the valve member, the mechanical collar having a selectively controlled inner diameter which can be manipulated to radially compress the valve member.

12. The hemostatic valve of claim 1 where the means for varying the diameter of the aperture and/or compression of the valve member around the aperture comprises at least one conical segment longitudinally disposable into the housing, the at least one conical segment being disposed about the valve member and serving to radially compress the valve member when longitudinally disposed into the housing.

13. The hemostatic valve of claim 1 where the means for varying the diameter of the aperture and/or compression of the valve member around the aperture comprises a plurality of nested conical segments longitudinally disposable into the housing, the nested conical segments being disposed about the valve member.

14. The hemostatic valve of claim 13 where the valve member is also conical in shape.

15. The hemostatic valve of claim 12 where the conical segment is oriented to have its least diameter proximally positioned in the housing.

16. The hemostatic valve of claim 14 where the conical segment is oriented to have its least diameter distally positioned in the housing.

17. The hemostatic valve of claim 1 where the valve member is a flexible funnel-shaped membrane defining the aperture therethrough, where the housing is comprised of a proximal and distal section, which are rotatable relative to each other, and where the means for varying the diameter of the aperture and/or compression of the valve member around the aperture comprises fixation of a proximal portion of the membrane to the proximal section of the housing and fixation of a distal portion of the membrane to the distal section of the housing to allow the membrane to be twisted into a funnel shape of variable aperture by relative rotation of the proximal and distal sections of the housing with respect to each other.

18. The hemostatic valve of claim 17 where the membrane has surface modifications defined therein which tend to define how the membrane folds when twisted to assume the funnel shape of variable aperture.

19. The hemostatic valve of claim 17 where the proximal and distal sections of the housing are threaded together with a pitch matched to the lengthening and shortening of the membrane as it is twisted in either direction.

20. The hemostatic valve of claim 1 where the valve member comprises a helical tube valve.

21. The hemostatic valve of claim 20 where the housing is comprised of a proximal and distal section, where the means for varying the diameter of the aperture and/or compression of the valve member around the aperture comprises means for telescopically displacing the proximal and distal sections with respect to each other.

22. A method of operating a separable hemostatic valve comprising:
inserting an elongate tool through or into an aperture defined in a separable valve member and separable valve housing;
applying a force to the valve member;
varying the diameter of the aperture and/or force closing the valve member around the elongate tool disposed in the aperture; and
separating the separable valve housing and/or separable valve member into at least two parts.

23. The method of claim 22 where varying the diameter of the aperture and/or compression of the valve member around the aperture comprises radially compressing the valve member.

24. The method of claim 22 where varying the diameter of the aperture and/or compression of the valve member around the aperture comprises longitudinally compressing the valve member.

25. The method of claim 1 where varying the diameter of the aperture and/or compression of the valve member around the aperture comprises laterally compressing the valve member.

26. The method of claim 22 where varying the diameter of the aperture and/or force closing the valve member comprises disposing a compression collar about a valve housing in which the valve member is disposed, which valve housing is compressed radially by the compression collar.

27. The method of claim 26 where the compression collar is substantially fixed in radius and is coupled to the housing, and where the housing has an inner diameter and walls which are of increasing radius, where disposing the compression collar longitudinally displaces the collar with respect to the housing to radially compress the housing walls together and to reduce the inner diameter of the housing thereby radially compressing the valve member therein and reducing the diameter of the aperture defined through the valve member.

28. The method of claim 22 where the housing has a substantially constant inner diameter and where varying the diameter of the aperture and/or compression of the valve member around the aperture comprises longitudinally disposing a collar within the inner diameter of the housing and where the collar has an inner diameter of decreasing magnitude as a function of distal position along the collar, so that longitudinal disposition of the collar into the housing compresses the valve member.

29. The method of claim 28 where the collar has a stepped inner diameter so that the valve member is compressed with one or more of a plurality of stepped forces.

30. The method of claim 22 where varying the diameter of the aperture and/or compression of the valve member around the aperture comprises disposing an hydraulic collar radially around the valve member, and inflating the hydraulic collar to radially compress the valve member.

31. The method of claim 22 where varying the diameter of the aperture and/or compression of the valve member around the aperture comprises disposing a mechanical collar radially around the valve member, the mechanical collar having a selectively controlled inner diameter, and manipulating the collar to radially compress the valve.member.

32. The method of claim 22 where varying the diameter of the aperture and/or compression of the valve member around the aperture comprises disposing at least one conical segment longitudinally into a housing about the valve member to radially compress the valve member.

33. The method of claim 22 where varying the diameter of the aperture and/or compression of the valve member around the aperture comprises longitudinally disposing a plurality of nested conical segments into a housing, the nested conical segments being disposed about the valve member.

34. The method of claim 33 where longitudinally disposing a plurality of nested conical segments into a housing comprises longitudinally disposing a plurality of nested conical segments about a conical valve member.

35. The method of claim 32 further comprising orienting the conical segment to have its least diameter proximally positioned in the housing.

36. The method of claim 32 further comprising orienting the conical segment to have its least diameter distally positioned in the housing.

37. The method of claim 22 where the valve member is a flexible funnel-shaped membrane defining the aperture therethrough, where the housing is comprised of a proximal and distal section, which are rotatable relative to each other, where a proximal portion of the membrane is fixed to the proximal section of the housing, where a distal portion of the membrane is fixed to the distal section of the housing, and where varying the diameter of the aperture and/or compression of the valve member around the aperture comprises rotating the proximal and distal sections of the housing with respect to each other to twist the membrane into a funnel shape of variable aperture.

38. The method of claim 29 further comprising folding the membrane when twisted to assume the funnel shape of variable aperture.

39. The method of claim 27 further comprising lengthening and shortening of the membrane as it is twisted in each respective direction when the proximal and distal sections of the housing are rotated relative to each other.

40. The method of claim 22 where varying the diameter of the aperture and/or compression of the valve member around the aperture comprises varying tension applied to a helical tube valve.

41. The method of claim 40 where the housing is comprised of a proximal and distal section, where varying the diameter of the aperture and/or compression of the valve member around the aperture comprises telescopically displacing the proximal and distal sections with respect to each other.
